(19) United States
(12) Patent Application Publication
(10) Pub. No.: US 2022/0327698 A1
(43) Pub. Date: Oct. 13, 2022

(54) X-RAY IMAGE PROCESSING APPARATUS, X-RAY DIAGNOSTIC APPARATUS, AND METHOD

(71) Applicant: CANON MEDICAL SYSTEMS CORPORATION, Tochigi (JP)

(72) Inventors: Yusuke OKUMURA, Nasushiobara (JP); Haruki IWAI, Otawara (JP); Motohiro SATO, Nasushiobara (JP)

(73) Assignee: CANON MEDICAL SYSTEMS CORPORATION, Tochigi (JP)

(21) Appl. No.: 17/658,857

(22) Filed: Apr. 12, 2022

(30) Foreign Application Priority Data
Apr. 12, 2021 (JP) 2021-067147

(51) Int. Cl. G06T 7/00; A61B 6/00

(52) U.S. Cl. CPC G06T 7/0012; A61B 6/487; A61B 6/5258; G06T 2207/30092; G06T 2207/10121; G06T 2200/24

(57) ABSTRACT

An X-ray image processing apparatus of an embodiment includes processing circuitry. The processing circuitry acquires fluoroscopy-related information indicating at least one of a fluoroscopic image and a condition for collecting the fluoroscopic image. The processing circuitry evaluates the image quality of the fluoroscopic image based on the fluoroscopy-related information. The processing circuitry outputs identification information identifying whether to save the fluoroscopic image based on the evaluation result.

### CROSS-REFERENCE TO RELATED APPLICATIONS

This application is based upon and claims the benefit of priority from Japanese Patent Application No. 2021-067147, filed on Apr. 12, 2021; the entire contents of which are incorporated herein by reference.

### FIELD

Embodiments described herein relate generally to an X-ray image processing apparatus, an X-ray diagnostic apparatus, and a method.

### BACKGROUND

In X-ray fluoroscopy, a user such as a radiologist waits for an image capturing timing of a subject while observing fluoroscopic images (moving images) of the subject continuously displayed on a display, and performs radiography on the subject by pressing down an exposure switch at the image capturing timing. In X-ray fluoroscopy, the subject is irradiated with a lower dose of X-rays than in radiography, and the fluoroscopic image is of lower quality than the captured image (still image) acquired by radiography is.

In recent years, image quality improvement technologies using artificial intelligence (AI) and other technologies have been improved, and high quality X-ray images can be acquired based on low quality X-ray images. If an X-ray image with an image quality that satisfies the diagnostic criteria (image quality that can be treated as a diagnostic image) is acquired by performing image quality improvement on the fluoroscopic image, the X-ray image may be saved as a diagnostic image, and collection of captured images becomes unnecessary, thereby reducing the exposure of the subject.

However, in a case where the image quality of the fluoroscopic image (the original X-ray image) is poor, it is not possible to acquire an X-ray image with image quality that satisfies the diagnostic criteria even when image quality improvement is performed on the fluoroscopic image. Therefore, the user needs to instantly determine whether to save the fluoroscopic image (whether an X-ray image with image quality equivalent to that of a captured image can be acquired by performing image quality improvement) or to perform radiography at the timing of saving the X-ray image (diagnostic image) while observing the fluoroscopic image of the subject displayed on the display. This places a heavy burden on the user.

### BRIEF DESCRIPTION OF THE DRAWINGS

FIG. 1 is a block diagram illustrating an example of a configuration of an X-ray diagnostic apparatus according to a first embodiment;

FIG. 2 is a flowchart illustrating a procedure of processing performed by the X-ray diagnostic apparatus according to the first embodiment;

FIG. 3A is a diagram for describing processing performed by each function according to the first embodiment;

FIG. 3B is a diagram for describing processing performed by each function according to the first embodiment;

FIG. 4A is a diagram illustrating an example of a display of an evaluation result according to the first embodiment;

FIG. 4B is a diagram illustrating an example of a display of an evaluation result according to the first embodiment;

FIG. 5 is a block diagram illustrating an example of a configuration of an X-ray diagnostic apparatus according to a second embodiment;

FIG. 6A is a diagram for describing processing performed by each function according to the second embodiment; and

FIG. 6B is a diagram for describing processing performed by each function according to the second embodiment.

### DETAILED DESCRIPTION

According to an embodiment, an X-ray image processing apparatus includes processing circuitry. The processing circuitry is configured to acquire fluoroscopy-related information indicating at least one of a fluoroscopic image and a condition for collecting the fluoroscopic image. The processing circuitry is configured to evaluate image quality of the fluoroscopic image based on the fluoroscopy-related information. The processing circuitry is configured to output identification information identifying whether to save the fluoroscopic image based on an evaluation result.

Hereinafter, details of embodiments of an X-ray image processing apparatus, an X-ray diagnostic apparatus, and a method will be described with reference to the accompanying drawings. Note that the X-ray image processing apparatus, the X-ray diagnostic apparatus, and the method according to the present application are not limited by the embodiments described below. In the following description, common reference signs are applied to similar structural components, and duplicate explanations are omitted.

### First Embodiment

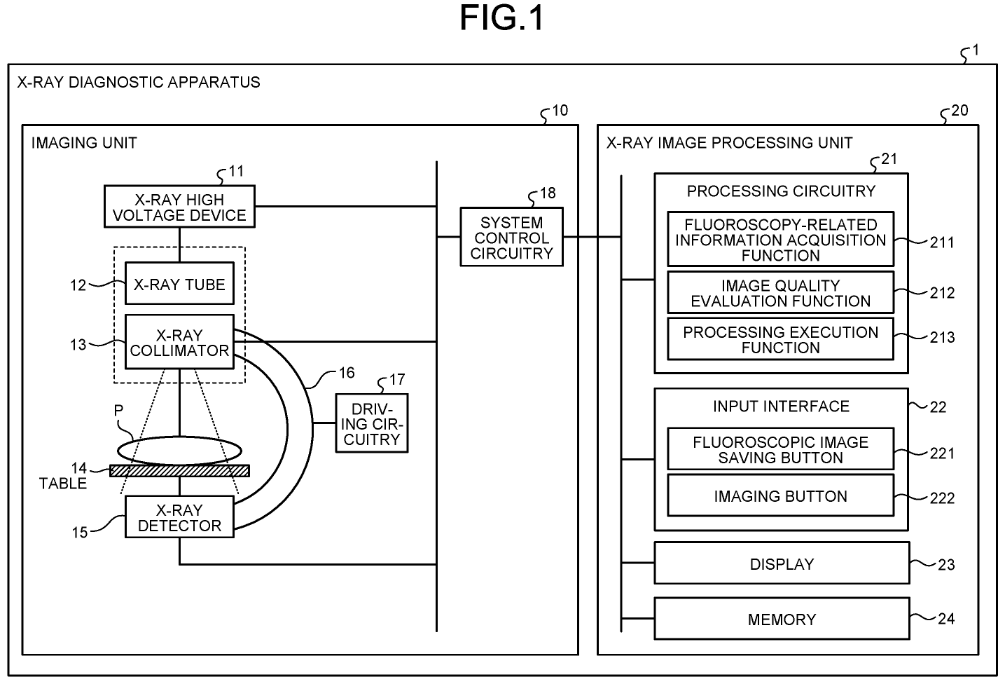

FIG. 1 is a block diagram illustrating an example of a configuration of an X-ray diagnostic apparatus 1 according to a first embodiment. As illustrated in FIG. 1, the X-ray diagnostic apparatus 1 includes an imaging unit 10 and an X-ray image processing unit 20.

The imaging unit 10 emits X-rays to a subject P and detects the X-rays transmitted through the subject P. The X-ray image processing unit 20 control the imaging unit 10 so that the imaging unit 10 emit X-rays, and performs image processing based on the X-rays detected by the imaging unit 10.

The imaging unit 10 includes an X-ray high voltage device 11, an X-ray tube 12, an X-ray collimator 13, a table 14, an X-ray detector 15, a C-arm 16, driving circuitry 17, and system control circuitry 18.

The X-ray high voltage device 11 generates a high voltage according to the control of the system control circuitry 18. The X-ray tube 12 emits X-rays based on the high voltage applied by the X-ray high voltage device 11. The X-ray collimator 13 includes two pairs of blades, each pair being provided on top and bottom as well as on left and right sides (four blades in total), for example. Each blade is formed in a flat plate shape by a material such as lead that blocks X-rays. The X-ray collimator 13 opens and closes the blades according to the control of the system control circuitry 18, and forms the irradiation range (irradiation field) of the X-rays emitted from the X-ray tube 12.

The table 14 is a bed where the subject P is placed on, and it is placed on top of a couch. The X-ray detector 15 is, for example, an X-ray flat panel detector (FPD) having detector elements arranged in matrix. The X-ray detector 15 detects the X-rays emitted from the X-ray tube 12 and transmitted through the subject P, and outputs a detection signal (X-ray detection signal) corresponding to the detected X-ray dose to processing circuitry 21.

The C-arm 16 holds the X-ray tube 12 and the X-ray collimator 13, and the X-ray detector 15 in a facing manner with the subject P in between. The driving circuitry 17 drives the C-arm 16 according to the control of the system control circuitry 18, and rotates and moves it with respect to the subject P.

The system control circuitry 18 is implemented by a processor, for example. The system control circuitry 18 receives control signals from the X-ray image processing unit 20 and controls the entire operation of the imaging unit 10 by controlling the X-ray high voltage device 11, the X-ray collimator 13, the X-ray detector 15, and the driving circuitry 17 based on the control signals.

The X-ray image processing unit 20 is an apparatus that processes an X-ray image and includes the processing circuitry 21, an input interface 22, a display 23, and a memory 24.

The input interface 22 is configured with an input device that accepts various input operations from the user such as a radiologist. The input interface 22 accepts input operations from the user and outputs electrical signals corresponding to the accepted input operations to the processing circuitry 21. For example, the input interface 22 includes a mouse, a keyboard, and a trackball. Furthermore, the input interface 22 includes a fluoroscopic image saving button 221 for giving an instruction to save fluoroscopic images and an imaging button 222 for capturing images, as operation buttons to accept operations from the user. Each of the fluoroscopic image saving button 221 and the imaging button 222 is configured with a hand switch (the exposure switch or the like), a foot switch, or the like, which accepts operations performed by the user's hand or foot. Furthermore, the input interface 22 may be configured with a touch pad for performing an input operation by touching an operation surface, a non-contact input circuit using an optical sensor, a voice input circuit, or the like.

The display 23 is configured with a display device that displays various kinds of information. For example, the display 23 displays X-ray images (fluoroscopic images and captured images) of the subject P collected based on X-ray fluoroscopy. Furthermore, the display 23 also displays a graphical user interface (GUI) and various kinds of information such as imaging conditions regarding X-ray images.

The memory 24 is configured with a semiconductor memory element such as a random-access memory (RAM) or a flash memory, a hard disk, an optical disc, or the like. The memory 24 stores therein various kinds of information used by and generated by the processing circuitry 21. For example, the memory 24 stores therein various kinds of information such as X-ray images of the subject P (fluoroscopic images and captured images) collected based on X-ray fluoroscopy, GUI, imaging conditions regarding X-ray images, and the like. Furthermore, the memory 24 stores therein a program that causes the processing circuitry 21 to function as a fluoroscopy-related information acquisition function 211, an image quality evaluation function 212, and a processing execution function 213.

The processing circuitry 21 is configured with a processor, for example. The processing circuitry 21 controls the entire X-ray diagnostic apparatus 1 by controlling each structural component of the X-ray image processing unit 20 and the system control circuitry 18 of the imaging unit 10. Specifically, the processing circuitry 21 supplies a control signal to the system control circuitry 18 to cause the imaging unit 10 to perform X-ray irradiation. The processing circuitry 21 receives a detection signal corresponding to the X-ray detected by the imaging unit 10 from the imaging unit 10, and generates an X-ray image.

Furthermore, the processing circuitry 21 reads out and executes the program stored in the memory 24 to function as the fluoroscopy-related information acquisition function 211, the image quality evaluation function 212, and the processing execution function 213. The processing circuitry 21 is an example of processing circuitry.

In the above, an example of the configuration of the X-ray diagnostic apparatus 1 according to the present embodiment has been described. Conventionally, in X-ray fluoroscopy, the user such as a radiologist waits for an image capturing timing (timing for collecting and saving diagnostic images) of a subject while observing fluoroscopic images (moving images) of the subject continuously displayed on a display, and performs radiography on the subject by pressing down an exposure switch at the image capturing timing. For example, in X-ray fluoroscopy of the stomach, low-dose X-rays are emitted to a subject that has taken barium (contrast medium), and fluoroscopic images (moving images) are continuously collected. While observing the flow and accumulation of the contrast medium from the fluoroscopic images of the subject displayed on the display, the user waits for the timing to capture an X-ray image (still image) to be saved as a diagnostic image, and performs an image capturing operation of the X-ray image at the image capturing timing. In X-ray fluoroscopy, the subject is irradiated with a lower dose of X-rays than in radiography, and the fluoroscopic image is of lower quality than the captured image (still image) acquired by radiography.

In recent years, image quality improvement technologies using artificial intelligence (AI) and other technologies have improved, and high quality X-ray images can be acquired based on low quality X-ray images. If an X-ray image with an image quality that satisfies diagnostic criteria (an image quality that can be treated as a diagnostic image, for example, an image quality equivalent to that of a radiographic image) can be acquired by performing image quality improvement such as noise reduction, super-resolution processing, high-resolution processing, and the like, the X-ray image can be saved as a diagnostic image, and collection of captured images becomes unnecessary. Thereby, the radiation exposure of the subject can be reduced.

However, in a case where the image quality of the fluoroscopic image (the original X-ray image) is poor, it is not possible to acquire an X-ray image with image quality that satisfies the diagnostic criteria even when image quality improvement is performed on the fluoroscopic image. Therefore, the user needs to instantly determine whether to save the fluoroscopic image (whether an X-ray image with image quality equivalent to that of a captured image can be acquired by performing image quality improvement) or to perform radiography at the timing of saving the X-ray image (diagnostic image) while observing the fluoroscopic image of the subject displayed on the display. This places a heavy burden on the user. In addition, applying image quality improvement to all frames (every frame) of the collected fluoroscopic images require a lot of processing load and time.

Therefore, the X-ray diagnostic apparatus 1 reduces the exposure of the subject P by suppressing the burden on the user such as a radiologist without performing image quality improvement on all frames (every frame) of the collected fluoroscopic images. The processing circuitry 21 of the X-ray diagnostic apparatus 1 performs (1) processing for acquiring fluoroscopy-related information regarding the fluoroscopic image as an evaluation target, (2) processing for evaluating the image quality of the fluoroscopic image as the evaluation target based on the acquired fluoroscopy-related information, and (3) processing based on the evaluation result of the image quality. Specifically, the processing circuitry 21 outputs identification information that identifies whether to save the fluoroscopic image based on the evaluation result of the image quality of the fluoroscopic image, and executes various kinds of processing corresponding to the identification information.

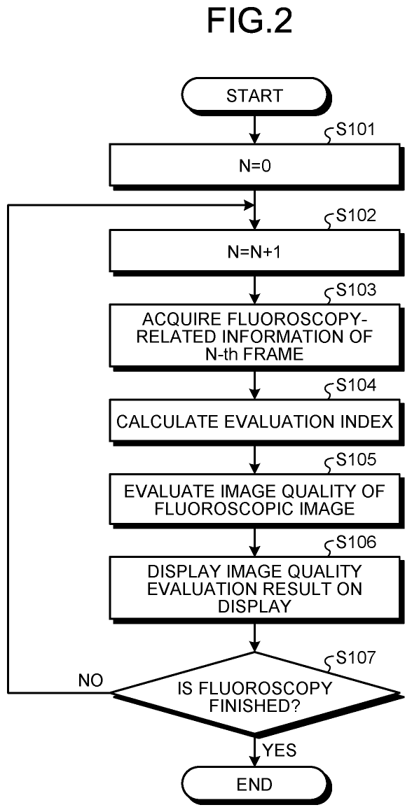

FIG. 2 is a flowchart illustrating a procedure of the processing performed by the processing circuitry 21 of the X-ray diagnostic apparatus 1. For example, when the user performs an operation to start collecting fluoroscopic images via the input interface 22 (for example, operation of stepping on a foot switch), the processing circuitry 21 reads out and executes the program stored in the memory 24 (start). Thereby, the processing circuitry 21 functions as the fluoroscopy-related information acquisition function 211, the image quality evaluation function 212, and the processing execution function 213.

The fluoroscopy-related information acquisition function 211 sets a frame number N of the fluoroscopic image to be collected to “N=0” (step S101), and then sets the frame number N to “N=N+1” (step S102). The fluoroscopy-related information acquisition function 211 acquires the fluoroscopy-related information of the N-th frame (step S103). In other words, after starting the execution of the program, the fluoroscopy-related information acquisition function 211 first acquires the fluoroscopy-related information of the first frame.

Fluoroscopy-related information is the fluoroscopic image of the N-th frame (the first frame in this case), for example. The fluoroscopy-related information acquisition function 211 receives the detection signal of the X-ray detected by the X-ray detector 15 of the imaging unit 10, generates the fluoroscopic image of the N-th frame based on the detection signal, and acquires it as the fluoroscopy-related information.

The fluoroscopy-related information is information related to the fluoroscopic image as the evaluation target. The fluoroscopic image as the evaluation target is a fluoroscopic image having improved image quality of the fluoroscopic image of the N-th frame (the first frame in this case), for example. As for the image quality improvement, there may be processing such as noise reduction, super-resolution processing, and high-resolution processing, for example, and those can be selected as desired. Noise reduction is the processing that reduces various noise components included in the fluoroscopic image. Super-resolution processing is the processing that generates a high-resolution fluoroscopic image based on a plurality of fluoroscopic images. High-resolution processing is the processing that generates a high-resolution fluoroscopic image by analyzing the features included in a single fluoroscopic image.

The image quality evaluation function 212 evaluates the image quality of a fluoroscopic image (fluoroscopic image as the evaluation target) having improved image quality of the fluoroscopic image of the N-th frame (the first frame in this case) based on the fluoroscopic image (fluoroscopy-related information). In other words, the image quality evaluation function 212 evaluates the image quality of the fluoroscopic image when the processing for improving the image quality is executed based on the fluoroscopic image of the N-th frame. The image quality evaluation function 212 calculates a predetermined evaluation index for the fluoroscopy-related information acquired by the fluoroscopy-related information acquisition function 211 (step S104). As the evaluation indices, there may be signal-to-noise ratio (SNR), contrast-to-noise ratio (CNR), a luminance value of the fluoroscopic image, and the like. For example, the image quality evaluation function 212 calculates the SNR of the fluoroscopic image of the N-th frame (the first frame in this case) acquired as the fluoroscopy-related information.

The image quality evaluation function 212 evaluates the image quality of the fluoroscopic image as the evaluation target based on the calculated evaluation index (step S105). For example, the image quality evaluation function 212 evaluates the image quality of the fluoroscopic image (the fluoroscopic image as the evaluation target) when the processing for improving the image quality is executed on the fluoroscopic image based on the calculated SNR.

Specifically, the image quality evaluation function 212 evaluates the image quality of the fluoroscopic image, and outputs identification information that identifies whether to save the fluoroscopic image according to the evaluation result. For example, the image quality evaluation function 212 outputs “1” as the identification information corresponding to the evaluation result when the calculated SNR exceeds a preset threshold, and outputs “0” as the identification information corresponding to the evaluation result when it is equal to or less than the threshold. The evaluation result of “1” indicates that the image quality of the fluoroscopic image (the fluoroscopic image as the evaluation target) after the image quality improvement satisfies the diagnostic criteria (can be treated as a diagnostic image). In other words, the evaluation result (identification information) of “1” indicates that the fluoroscopic image can be saved. The evaluation result of “0” indicates that the image quality of the fluoroscopic image after the image quality improvement does not satisfy the diagnostic criteria (cannot be treated as a diagnostic image). In other words, the evaluation result (identification information) of “0” indicates that the fluoroscopic image cannot be saved.

The threshold is a standard value that evaluates whether a fluoroscopic image, when the processing for improving the image quality is executed thereon, can be a fluoroscopic image having the image quality that satisfies the diagnostic criteria (image quality that can be treated as a diagnostic image). For example, for the X-ray image that comes to have the image quality satisfying the diagnostic criteria by execution of the processing for improving the image quality, the threshold is set based on the SNR of the X-ray image before the image quality improvement. As described, the image quality evaluation function 212 evaluates the image quality of the fluoroscopic image (fluoroscopic image as the evaluation target) having the improved image quality of the fluoroscopic image of the N-th frame (the first frame in this case) based on the fluoroscopic image (fluoroscopy-related information) of the N-th frame (the first frame in this case).

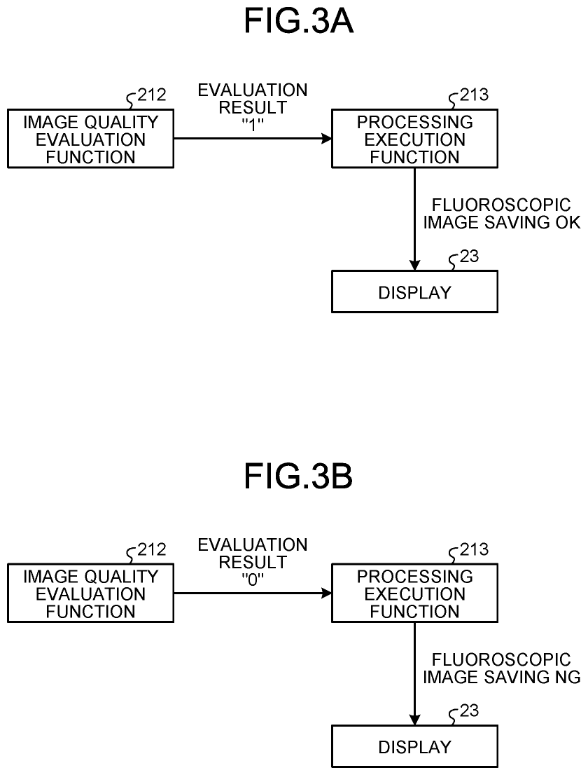

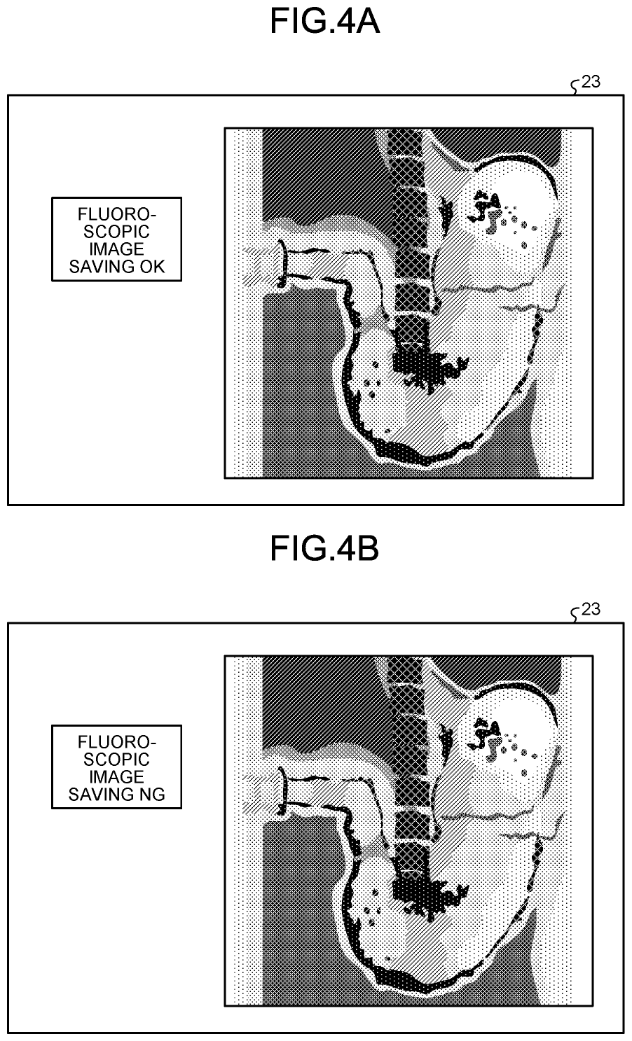

The processing execution function 213 displays the information based on the identification information according to the evaluation result output by the image quality evaluation function 212 on the display 23 together with the fluoroscopic image of the N-th frame (the first frame in this case) generated by the fluoroscopy-related information acquisition function 211 (step S106). Specifically, as illustrated in FIG. 3A and FIG. 4A, when the image quality evaluation function 212 outputs the identification information “1” corresponding to the evaluation result, the processing execution function 213 displays the information identifying that the fluoroscopic image can be saved (fluoroscopic image saving OK) together with the fluoroscopic image. In the meantime, as illustrated in FIG. 3B and FIG. 4B, when the image quality evaluation function 212 outputs the identification information “0” corresponding to the evaluation result, the processing execution function 213 displays the information identifying that the fluoroscopic image cannot be saved (fluoroscopic image saving NG) together with the fluoroscopic image. FIG. 3A and FIG. 3B are diagrams for describing the processing performed by each function according to the first embodiment. FIG. 4A and FIG. 4B are diagrams illustrating examples of a display of the evaluation result according to the first embodiment.

Based on this information, the user can confirm whether to save the fluoroscopic image displayed on the display 23. Note that the information identifying whether to save the fluoroscopic image may be expressed by light, sound, vibration, or the like other than text. For example, the processing execution function 213 may emit green light when the fluoroscopic image can be saved, and may emit red light when the fluoroscopic image cannot be saved. Note that the information identifying whether the fluoroscopic image can be saved may be displayed on the display 23 only in either one of the cases where the fluoroscopic image can be saved or where the fluoroscopic image cannot be saved. For example, the frame line of the fluoroscopic image may be caused to emit green light when the fluoroscopic image can be saved, and the frame line of the fluoroscopic image may be caused not to emit light when the fluoroscopic image cannot be saved.

Then, the processing execution function 213 determines whether collection of fluoroscopic images is finished (step S107). For example, the processing execution function 213 determines whether collection of fluoroscopic images is finished based on whether an operation for ending the collection of fluoroscopic images (for example, an operation to release the foot switch) is received via the input interface 22. When no operation for ending collection of fluoroscopic images is received (No at step S107), the processing execution function 213 performs the same processing as above for the fluoroscopic image of the next frame (the second frame in this case) (steps S102 to S107). The processing execution function 213 repeats a series of the processing (steps 5102 to S107) for each frame until an operation for ending collection of the fluoroscopic images is received. In the meantime, when there is an operation for ending collection of fluoroscopic images is received (Yes at step S107), the processing execution function 213 ends the processing (end).

As described above, with the X-ray diagnostic apparatus 1 according to the first embodiment, the fluoroscopy-related information acquisition function 211 acquires the fluoroscopy-related information related to the fluoroscopic image as the evaluation target. The image quality evaluation function 212 evaluates the image quality of the fluoroscopic image as the evaluation target based on the fluoroscopy-related information. The processing execution function 213 executes the processing based on the image quality evaluated by the image quality evaluation function 212. The X-ray diagnostic apparatus 1 reduces the exposure of the subject P by suppressing the burden on the user such as a radiologist without performing image quality improvement on all frames (every frame) of the collected fluoroscopic images.

In addition, since the X-ray diagnostic apparatus 1 displays information identifying whether to save the fluoroscopic image (fluoroscopic image saving OK or fluoroscopic image saving NG) on the display 23 together with the fluoroscopic image, the user can promptly and easily determine whether the fluoroscopic image can be saved and whether collection of captured images is necessary by checking the information identifying whether to save the fluoroscopic image displayed on the display 23. Therefore, the user can appropriately operate the fluoroscopic image saving button 221 and the imaging button 222.

While the first embodiment is described by referring to an example in which acquisition of fluoroscopy-related information, evaluation of the image quality of the fluoroscopic image as the evaluation target, and execution of the processing based on the evaluated image quality are performed for each frame of continuously collected fluoroscopic images, such processing may be performed for every plural frames. For example, when the fluoroscopic image is displayed on the display 23 at a frame rate of 15 fps, acquisition of the fluoroscopy-related information, evaluation of the image quality of the fluoroscopic image as the evaluation target, and execution of the processing based on the evaluated image quality may be performed every 15 frames. In this case, the information identifying whether to save the fluoroscopic image displayed on the display 23 may be updated at 1-second intervals or deleted after being displayed for a predetermined time (for example, after 0.5 seconds).

Furthermore, while the first embodiment is described by referring to an example where the processing execution function 213 displays the information based on the evaluation result output by the image quality evaluation function 212 on the display 23 together with the fluoroscopic image of the N-th frame generated by the fluoroscopy-related information acquisition function 211, the fluoroscopic image and the information based on the evaluation result may be displayed on the display 23 at different timings. For example, the processing execution function 213 may set a lower display rate of the evaluation index for the fluoroscopic image than the display rate of the fluoroscopic image. In that case, after the fluoroscopic image is displayed on the display 23, the information based on the evaluation result for the fluoroscopic image is displayed on the display 23.

Furthermore, when the fluoroscopic image saving button 221 is pressed down while information indicating that the fluoroscopic image can be saved (fluoroscopic image saving OK) is displayed on the display 23, the processing execution function 213 may perform image quality improvement (for example, processing such as noise reduction, super-resolution processing, high-resolution processing) on the fluoroscopic image (relevant fluoroscopic image) displayed on the display 23 and store the fluoroscopic image with the improved image quality in the memory 24. Thereby, an X-ray image having the image quality that satisfies the diagnostic criteria (image quality that can be treated as a diagnostic image) can be acquired, so that collection of captured images is no longer necessary. Therefore, the exposure of the subject can be reduced. Note that the image quality improvement may be performed using AI such as a machine-learned trained model or rule-based AI, or may be performed by arithmetic processing without using AI.

Furthermore, when the fluoroscopic image saving button 221 is pressed down while information indicating that the fluoroscopic image can be saved (fluoroscopic image saving OK) is displayed on the display 23, the processing execution function 213 may store, in the memory 24, the fluoroscopic image displayed on the display 23 (relevant fluoroscopic image) by attaching the information identifying that the image quality improvement is to be performed after the fluoroscopy is completed. In this case, the processing circuitry 21 may perform image quality improvement (for example, processing such as noise reduction, super-resolution processing, high-resolution processing) on the fluoroscopic image (relevant fluoroscopic image) at any timing, such as when the fluoroscopic image is read out from the memory 24 or at predetermined date and time, and store the fluoroscopic image with the improved image quality in the memory 24. Thereby, an X-ray image having the image quality that satisfies the diagnostic criteria (image quality that can be treated as a diagnostic image) can be acquired, so that collection of captured images is no longer necessary. Therefore, the exposure of the subject can be reduced.

When the imaging button 222 is pressed down while the information indicating that the fluoroscopic image cannot be saved (fluoroscopic image saving NG) is displayed on the display 23, the processing execution function 213 performs radiography on the subject P and stores the captured image (X-ray image) acquired by radiography in the memory 24.

Furthermore, while the first embodiment is described by referring to an example where the fluoroscopic image of the N-th frame is acquired as the fluoroscopy-related information, various conditions set for collecting the fluoroscopic images may also be acquired as the fluoroscopy-related information. In addition, the image quality of the fluoroscopic image with the improved image quality (fluoroscopic image as the evaluation target) may be evaluated based on various acquired conditions, and information identifying whether the fluoroscopic image can be saved (fluoroscopic image saving OK or fluoroscopic image saving NG) may be displayed on the display 23 based on the evaluation result of the image quality.

The various conditions include an X-ray condition, a geometric condition, a control condition, and the like. The X-ray condition is the condition related to the X-rays emitted to the subject P, including tube voltage, tube current, focal spot size, radiation-quality filter, dose, pulse width, and the like. The geometric condition is the condition that indicates the positional relationship between the X-ray tube 12, the subject P, and the X-ray detector 15, such as the distance between the X-ray tube 12 and the X-ray detector 15 (source image distance: SID), for example. The control condition is the information that controls the X-ray tube 12 and the couch.

For example, the image quality evaluation function 212 compares the acquired condition with a threshold set in advance for that condition, and outputs “1” as the evaluation result when the result exceeds the threshold, and outputs “0” as the evaluation result when the result is equal to or less than the threshold. For example, for the X-ray image that comes to have an image quality satisfying the diagnostic criteria by executing the processing for improving the image quality, the threshold is set based on the condition at the time of collecting the X-ray images before the image quality improvement.

Furthermore, the evaluation index may be acquired not from the fluoroscopic image itself but from the various conditions. For example, the image quality evaluation function 212 may calculate the SNR (evaluation index) of the fluoroscopic image based on the X-ray condition acquired as fluoroscopy-related information. In such a case, the image quality evaluation function 212 calculates the SNR using, for example, the correspondence between the X-ray condition of the fluoroscopic image and the SNR estimated for that fluoroscopic image mapped in advance. Note here that the above correspondence may be constructed by machine learning. Then, as in the first embodiment, the image quality evaluation function 212 outputs “1” as an evaluation result when the calculated SNR exceeds a preset threshold, and outputs “0” as an evaluation result when it is equal to or less than the threshold. The same can be performed when the above setting conditions other than X-ray condition are used as the fluoroscopy-related information, or when the above evaluation indices other than the SNR are defined.

Furthermore, various kinds of information acquired at the time of collecting fluoroscopic images may be acquired as the fluoroscopy-related information. In addition, the image quality of the fluoroscopic image with the improved image quality (fluoroscopic image as the evaluation target) may be evaluated based on various kinds of acquired information, and information identifying whether the fluoroscopic image can be saved (fluoroscopic image saving OK or fluoroscopic image saving NG) may be displayed on the display 23 based on the evaluation result of the image quality.

The various kinds of information include the detection signal of the X-ray detected by the X-ray detector 15, irradiation dose to the subject P, incident dose to the X-ray detector 15 (estimated value or measured value), dose index (exposure index (EI) value or deviation index (DI) value), state of the subject P (shift amount of the subject P between fluoroscopic images), and the like.

For example, the image quality evaluation function 212 compares the acquired information with a threshold set in advance for the information, and outputs “1” as the identification information corresponding to the evaluation result when the result exceeds the threshold, and outputs “0” as the identification information corresponding to the evaluation result when the result is equal to or less than the threshold. For example, for the X-ray image that comes to have an image quality satisfying the diagnostic criteria by executing the processing for improving the image quality, the threshold is set based on the information at the time of collecting the X-ray images before the image quality improvement.

Furthermore, the evaluation index may be acquired not from the fluoroscopic image itself but from the various kinds of information. For example, the image quality evaluation function 212 may calculate the SNR (evaluation index) of the fluoroscopic image based on the irradiation dose to the subject P acquired as the fluoroscopy-related information. In such a case, the image quality evaluation function 212 calculates the SNR using, for example, information on the SNR for each irradiation dose mapped in advance. Then, as in the first embodiment, the image quality evaluation function 212 outputs “1” as the identification information corresponding to the evaluation result when the calculated SNR exceeds the preset threshold, and outputs “0” as the identification information corresponding to the evaluation result when it is equal to or less than the threshold. The same can be implemented when the above collected information other than the irradiation dose to the subject P is used as the fluoroscopy-related information or when the above evaluation indices other than the SNR are defined.

Note that the evaluation index may be the fluoroscopy-related information itself. For example, the X-ray condition acquired as the fluoroscopy-related information may be used as an evaluation index, and the image quality evaluation function 212 may output an evaluation result of “1” or “0” based on a comparison between the X-ray condition and the corresponding threshold. Furthermore, when the shift amount of the subject P is large between fluoroscopic images, blurring due to the movement of the subject P may be generated between the fluoroscopic images. Therefore, the state of the subject P (the shift amount of the subject P between the fluoroscopic images) may be used as an evaluation index, and the image quality evaluation function 212 may output the evaluation result of “1” or “0” based on a comparison between the state of the subject P and a corresponding threshold.

The threshold corresponding to the evaluation index may be set based on whether to save the fluoroscopic image determined by the user for the fluoroscopic image having the improved image quality (the fluoroscopic image as the evaluation target). For example, when the irradiation dose is used as the evaluation index, image quality improvement is performed for each of the fluoroscopic images acquired by irradiating the subject with different irradiation doses a plurality of times, and the user determines whether to save each of the fluoroscopic images having the improved image quality (fluoroscopic images as the evaluation target). For the two fluoroscopic images for which the determination result by the user changes from “saving OK” to “saving NG”, the intermediate value or the like of the irradiation doses corresponding to the images may be set as the threshold corresponding to the irradiation dose (evaluation index).

While the first embodiment is described by referring to an example where the evaluation result of “1” or “0” is output based on a comparison result between the evaluation index and the preset threshold, an evaluation for the evaluation index may be performed using AI such as a machine-learned trained model or rule-based AI, and the evaluation result may be output. For example, the image quality evaluation function 212 may input the fluoroscopy-related information or the evaluation indices to the trained model described above, and acquire the evaluation result of “1” or “0” that is acquired as the output result of the trained model.

Furthermore, the fluoroscopy-related information acquisition function 211 may acquire a combination of a plurality of kinds of fluoroscopy-related information, and the image quality evaluation function 212 may comprehensively evaluate the image quality of the fluoroscopic image as the evaluation target based on such kinds of fluoroscopy-related information. In other words, such kinds of fluoroscopy-related information, which are selectively combined from the fluoroscopic image of the N-th frame, various conditions set for collecting the fluoroscopic images, and various kinds of information acquired when the fluoroscopic images are collected, may be acquired, and the image quality of the fluoroscopic image as the evaluation target may be comprehensively evaluated based on the evaluation result corresponding to each of such kinds of fluoroscopy-related information.

When the image quality evaluation function 212 outputs an evaluation result of “0,” that is, when the fluoroscopic image cannot be saved, the processing execution function 213 may display a message on the display 23 prompting the user to change the various conditions. For example, when the image quality evaluation function 212 outputs an evaluation result of “0” continuously for a predetermined number of times, the processing execution function 213 displays a message on the display 23 recommending that any of the X-ray condition, the geometry condition, and the control condition be changed.

Furthermore, the processing execution function 213 may perform automatic brightness control (ABC) based on the evaluation result of the image quality acquired by the image quality evaluation function 212. Note that ABC is a control that feeds back the statistics of pixel values in the fluoroscopic image (for example, the average value of pixels in the region of interest) to the X-ray condition for the fluoroscopy of the next frame to bring the statistics closer to the target value. Specifically, the processing execution function 213 defines the target value of the pixel value of the fluoroscopic image as the luminance value of the fluoroscopic image that is determined to allow to be saved, and executes ABC using the determination result for the fluoroscopic image. For example, when determined that the fluoroscopic image as the evaluation target cannot be saved, the processing execution function 213 sets the luminance value determined to allow to be saved as the target and changes the X-ray condition of fluoroscopy to increase the dose. In the meantime, when determined that the fluoroscopic image as the evaluation target can be saved, the processing execution function 213 executes the existing ABC.

The image quality evaluation function 212 may also evaluate the image quality of the fluoroscopic image as the evaluation target according to the type of image quality improvement processing that reduces noise. Specifically, the processing execution function 213 may define the evaluation criteria in accordance with the characteristics of each type of image processing applied to the fluoroscopic images when performing image quality improvement. For example, when image processing that excels at reducing noise is performed on the fluoroscopic image as the saving target, the image quality evaluation function 212 changes the threshold corresponding to the evaluation index of image quality to be lower than usual. This allows the processing execution function 213 to determine even a fluoroscopic image with somewhat poor image quality to allow to be saved, and to generate a fluoroscopic image that can be used as a diagnostic image by executing image processing that excels at reducing noise. The X-ray diagnostic apparatus 1 can generate a fluoroscopic image having improved image quality from a fluoroscopic image, so that it is possible to reduce exposure to radiation. The image processing for which the processing execution function 213 defines the evaluation criteria for image quality in accordance with the characteristics is not limited to one kind. For example, also when the processing execution function 213 performs noise reduction using a plurality of kinds of image processing, the evaluation criteria for image quality may be defined in accordance with the characteristics of the entire image processing of a plurality of kinds.

Furthermore, the processing execution function 213 may enable one of the fluoroscopic image saving button 221 and the imaging button 222, and disable the other based on the results evaluated by the image quality evaluation function 212. For example, when the evaluation result acquired by the image quality evaluation function 212 is “1”, it indicates that the image quality of the fluoroscopic image after image quality improvement (the fluoroscopic image as the evaluation target) satisfies the diagnostic criteria. Therefore, the processing execution function 213 enables the fluoroscopic image saving button 221 and disables the imaging button 222. In the meantime, when the evaluation result acquired by the image quality evaluation function 212 is “0”, it indicates that the image quality of the fluoroscopic image after image quality improvement (the fluoroscopic image as the evaluation target) does not satisfy the diagnostic criteria. Therefore, the processing execution function 213 disables the fluoroscopic image saving button 221 and enables the imaging button 222. This can prevent unnecessary exposure of the subject P even when the user accidentally operates the fluoroscopic image saving button 221 and the imaging button 222.

### Second Embodiment

While the first embodiment is described by referring to an example where the two operation buttons for fluoroscopic image saving (fluoroscopic image saving button 221) and for capturing image (imaging button 222) are independent from each other, a second embodiment will be described by referring to an example of a case where a single operation button is shared between fluoroscopic image saving and image capturing.

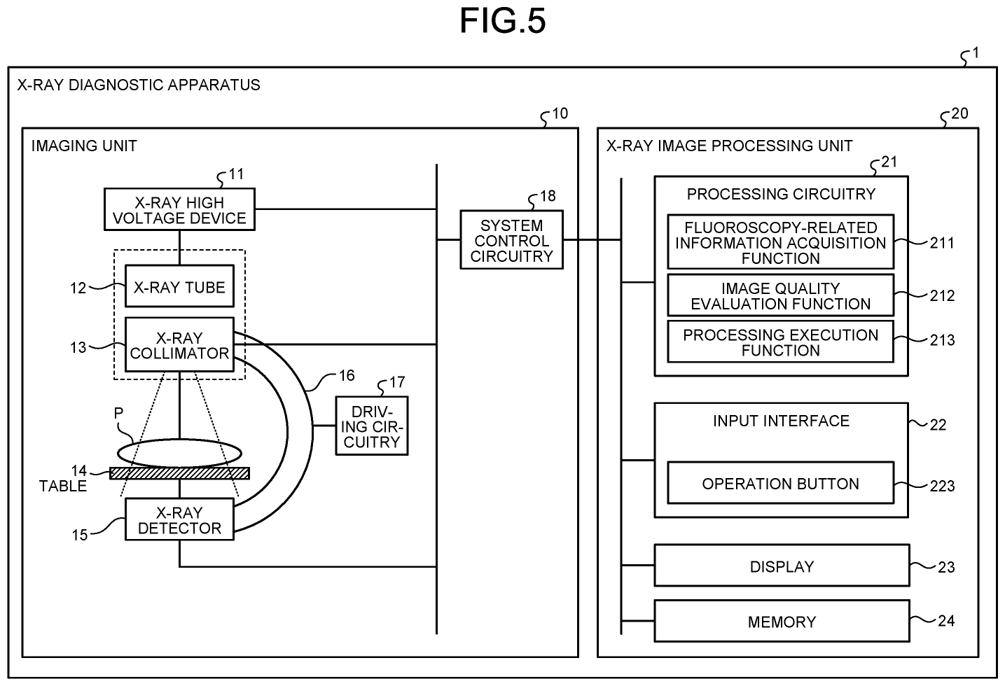

FIG. 5 is a block diagram illustrating an example of a configuration of an X-ray diagnostic apparatus according to the second embodiment. As illustrated in FIG. 5, the input interface 22 includes a single operation button 223. The processing execution function 213 selectively switches the operation button 223 to either the function for fluoroscopic image saving or for capturing image, based on the evaluation result of image quality performed by the image quality evaluation function 212.

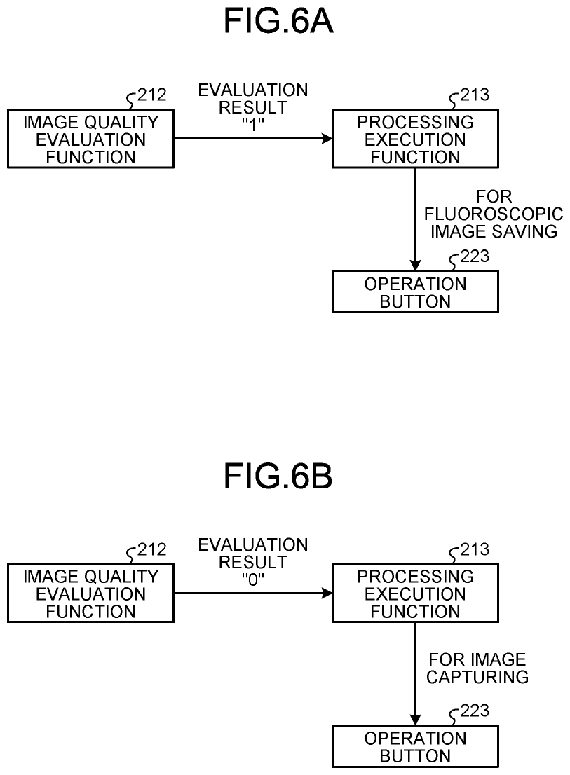

Specifically, as illustrated in FIG. 6A, when the image quality evaluation function 212 outputs “1” as the identification information corresponding to the evaluation result, the processing execution function 213 controls the operation button 223 to function for fluoroscopic image saving. Furthermore, as illustrated in FIG. 6B, when the image quality evaluation function 212 outputs “0” as the identification information corresponding to the evaluation result, the processing execution function 213 controls the operation button 223 to function for capturing image. FIG. 6A and FIG. 6B are diagrams for describing the processing performed by each function according to the second embodiment.

As described above, with the X-ray diagnostic apparatus 1 according to the second embodiment, the processing execution function 213 controls the operation button 223 that receives a user operation to function whether for fluoroscopic image saving or for capturing image based on the image quality evaluated by the image quality evaluation function 212. As a result, the user can save or capture the fluoroscopic image appropriately by simply waiting for the timing for saving the X-ray image (diagnostic image) while observing the fluoroscopic images (moving images) of the subject P continuously displayed on the display 23, and by pressing the operation button 223 at the timing. This makes it possible to suppress the burden on the user and reduce exposure.

In the first and second embodiments, the fluoroscopic image having the improved image quality is to be the fluoroscopic image as the evaluation target on assumption that image quality improvement is to be performed on the fluoroscopic image. However, the fluoroscopic image itself displayed on the display 23 (the fluoroscopic image of the N-th frame) may also be used as the fluoroscopic image as the evaluation target. Also, when the image quality of the fluoroscopic image itself satisfies the diagnostic criteria, such as a case where the fluoroscopic image itself can be treated as a diagnostic image as it is, there is no need to improve the image quality of the fluoroscopic image. For example, the fluoroscopy-related information acquisition function 211 acquires various conditions set for collecting fluoroscopic images (X-ray condition, geometric condition, control condition, and the like) and various kinds of information acquired when the fluoroscopic images are collected (irradiation dose to the subject P, incident dose to the X-ray detector 15 (estimated value or measured value), dose index (EI value or DI value), the state of the subject P (shift amount of the subject P between fluoroscopic images), and the like) are acquired as the fluoroscopy-related information. The image quality evaluation function 212 evaluates the image quality of the fluoroscopic image itself (the fluoroscopic image as the evaluation target) based on the fluoroscopy-related information, and the processing execution function 213 executes processing based on the image quality evaluated by the image quality evaluation function 212 (for example, displaying fluoroscopic image saving OK or NG, and switching the functions of the operation button 223).

Note here that the processing execution function 213 can distinguish and display the fluoroscopic images between a case where the fluoroscopic image itself can be treated as a diagnostic image as it is and a case where it can be treated as a diagnostic image by performing image quality improvement, as a result of the evaluation of the fluoroscopic images. For example, when the fluoroscopic image itself can be treated as a diagnostic image as it is, the processing execution function 213 displays information indicating the effect.

The first and second embodiments are described by referring to a case where the user operates the operation button when saving the fluoroscopic image or collecting the captured images. However, the embodiments are not limited thereto. After the image quality of the fluoroscopic images collected as the evaluation target is evaluated, the fluoroscopic images may be saved based on the evaluation result of the image quality without the button operations. For example, the image quality evaluation function 212 evaluates the image quality of sequentially collected fluoroscopic images. The processing execution function 213 extracts the fluoroscopic images that are determined to allow to be saved based on the evaluation result of image quality, and performs image quality improvement. Then, the processing execution function 213 saves the generated diagnostic images in the memory 24. This allows the user to save the fluoroscopic images without operating the operation button while the user performs a fluoroscopy instruction such as stepping on the foot switch. Therefore, fluoroscopic images can be saved without increasing the number of operations performed by the user, so that it is possible to suppress the burden on the user and reduce exposure.

Note here that the timing of generating diagnostic images is not limited to immediately after whether to save the images is determined. The processing execution function 213 may save the fluoroscopic images extracted to allow to be saved in the memory 24, and perform the image processing at any timing such as after the examination is finished. Furthermore, in a case where the extracted fluoroscopic images include the fluoroscopic image that has the image quality as a diagnostic image, the processing execution function 213 may omit the image quality improvement for such a fluoroscopic image.

The first and second embodiments are described by referring to a case where the parameters of the image processing such as noise reduction are not changed for the fluoroscopic image that is determined to allow be saved. However, the embodiments are not limited thereto, and the processing execution function 213 may change the parameters of the image processing according to the evaluation result of image quality when performing image quality improvement for the fluoroscopic images. Specifically, when evaluating the image quality by calculating the SNR for the fluoroscopic image, the processing execution function 213 changes the parameters for image quality improvement according to a difference between the calculated SNR and the threshold. For example, in a case where the image quality of the fluoroscopic image is considerably better than the image quality defined as the standard by the threshold, and the difference between the SNR and the threshold is large, the processing execution function 213 can decrease the extent of image quality improvement to be applied. In this case, the processing execution function 213 may change the parameters of the image quality improvement and execute the image processing to the extent necessary to generate the diagnostic image.

Furthermore, in the first and second embodiments, image quality improvement is performed on the fluoroscopic images saved in response to the operation of the user to generate diagnostic images. However, the embodiments are not limited thereto. When a plurality of fluoroscopic images can be saved, the processing execution function 213 may not perform image quality improvement for all fluoroscopic images that can be saved but may perform image quality improvement only for the fluoroscopic image designated by the user from the fluoroscopic images that can be saved. This allows the user to generate a diagnostic image based on the fluoroscopic image determined to be most suitable for generating the diagnostic image.

The processing execution function 213 can also perform the control to save fluoroscopic images in sequence imaging (for example, long imaging, tomosynthesis imaging, or the like) in which a plurality of captured images are collected by rotating and moving the X-ray tube 12 relative to the subject P. In other words, when the evaluation results of the image quality of the fluoroscopic images collected prior to sequence imaging can be saved, the processing execution function 213 performs image collection similar to sequence imaging under a fluoroscopy condition in response to the pressing of the imaging button 222 for executing sequence imaging, and saves a plurality of fluoroscopic images. The processing execution function 213 then generates a plurality of diagnostic images by executing image quality improvement on the collected fluoroscopic images, and generates a long image and a tomosynthesis image. The image quality improvement for generating a tomosynthesis image may be executed on a plurality of fluoroscopic images before reconstruction, or on a three-dimensional image reconstructed from a plurality of fluoroscopic images. This allows the X-ray diagnostic apparatus 1 to avoid capturing images by suppressing burden on the user and to reduce exposure even in sequence imaging other than normal imaging.

Since the evaluation index value required for the image quality of diagnostic images differs depending on the type of imaging to be performed, such as long imaging or tomosynthesis imaging, the image quality evaluation function 212 may change the threshold for evaluating the image quality of the fluoroscopic image beforehand according to the type of imaging.

In addition, the processing execution function 213 can change the time until collecting the fluoroscopic images in accordance with the delay time required from pressing of the imaging button 222 until capturing an image. Specifically, when the processing execution function 213 determines that a fluoroscopic image can be saved and accepts pressing of the imaging button 222 performed by the operator, a delay time is provided for collecting the fluoroscopic images as the saving target such that the fluoroscopic images are collected at the same timing as the normal imaging timing. This makes it possible to save the fluoroscopic images collected at the same timing as the captured images, and deviations from the user's desired timing can be suppressed even when the fluoroscopic images are saved.

The processing execution function 213 may continue to collect fluoroscopic images or may stop collecting fluoroscopic images between pressing of the imaging button 222 and saving the fluoroscopic image. When collection of the fluoroscopic images is to be continued, the processing execution function 213 can also take the fluoroscopic images collected at the same (or approximate) timing as (to) the captured images as the saving target without the delay time. This makes it possible to suppress deviations from the user's desired timing without providing the delay time when collecting the fluoroscopic images. Furthermore, the processing execution function 213 may display the collected fluoroscopic images on the display 23 when collecting the fluoroscopic images. This enables the X-ray diagnostic apparatus 1 to observe fluoroscopic images collected sequentially during the examination in real time.

The first and second embodiments are described by referring to a case where the display image is used to evaluate the image quality of the fluoroscopic image. However, the embodiments are not limited thereto; and the detection signal may be used to evaluate the image quality of the fluoroscopic image, for example.

In addition, the term “processor” used in the above description of the embodiments means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)). Here, instead of saving the program in the memory circuit, the program can be directly incorporated in the circuit of the processor. In this case, the processor implements the functions by reading out and executing the program incorporated in the circuit. Each of the processors of the present embodiment is not limited to being configured as a single circuit for each processor, but may also be configured as a single processor by combining a plurality of independent circuits to implement the functions.

Note here that the program to be executed by the processor is provided by being incorporated in advance in a read-only memory (ROM), a memory circuit, or the like. The program may be provided in a file of format that can be installed on such devices or in an executable format by being recorded on a computer-readable non-transitory storage medium such as a compact disc (CD)-ROM, a flexible disk (FD), a CD-R (recordable), a digital versatile disc (DVD), or the like. Furthermore, the program may also be stored on a computer connected to a network such as the Internet, and provided or distributed by being downloaded via the network. For example, the program is configured with modules including the processing functions described above. As for the actual hardware, the CPU reads out and executes the program from a storage medium such as a ROM, so that each of the modules is loaded onto the main memory and generated on the main memory.

Furthermore, in the embodiments described above, each of the structural components of each of the illustrated devices is a functional concept and does not necessarily need to be physically configured as illustrated in the drawings. In other words, the specific form of distribution or integration of the devices is not limited to those illustrated in the drawings, but all or some of the devices can be physically distributed or integrated in an arbitrary unit in accordance with various kinds of load, use state, or the like. Furthermore, all or some of the processing functions performed by respective devices can be implemented by the CPU and the program that is analyzed and executed by the CPU, or may be implemented by hardware using wired logic.

Among the pieces of processing described in the above embodiments, all or some of them described to be performed automatically can be performed manually, or all or some of them described to be performed manually can be performed automatically using a known method. In addition to the above, the processing procedures, control procedures, specific names, and information including various kinds of data and parameters discussed in the description and drawings can be changed arbitrarily, except as otherwise noted.

According to at least one of the embodiments described above, it is possible to suppress burden on the user and reduce exposure.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray image processing apparatus comprising processing circuitry configured to
acquire fluoroscopy-related information indicating at least one of a fluoroscopic image and a condition for collecting the fluoroscopic image,
evaluate image quality of the fluoroscopic image based on the fluoroscopy-related information, and
output identification information identifying whether to save the fluoroscopic image based on an evaluation result.

2. The X-ray image processing apparatus according to claim 1, wherein the processing circuitry is configured to display information identifying whether to save the fluoroscopic image on a display based on the identification information.

3. The X-ray image processing apparatus according to claim 1, wherein the processing circuitry is configured to select whether an input interface receiving an operation of a user is to be used for fluoroscopic image saving or for image capturing based on the identification information.

4. The X-ray image processing apparatus according to claim 1, wherein the processing circuitry is configured to execute image processing for improving the image quality of the fluoroscopic image based on the identification information.

5. The X-ray image processing apparatus according to claim 1, wherein the processing circuitry is configured to accept an operation for changing the condition for collecting the fluoroscopic image based on the identification information.

6. The X-ray image processing apparatus according to claim 1, wherein the processing circuitry is configured to calculate a predetermined evaluation index of the fluoroscopic image as an evaluation target, and evaluate the image quality of the fluoroscopic image as the evaluation target by the calculated evaluation index.

7. The X-ray image processing apparatus according to claim 1, wherein the processing circuitry is configured to evaluate the image quality of the fluoroscopic image as an evaluation target according to a type of image processing performed for noise reduction.

8. An X-ray diagnostic apparatus comprising:
an X-ray tube configured to emit an X-ray;
an X-ray detector configured to detect the X-ray emitted by the X-ray tube; and
an X-ray image processing apparatus configured to process an X-ray image based on the X-ray detected by the X-ray detector, wherein
the X-ray image processing apparatus comprises a processing circuitry configured to
acquire fluoroscopy-related information indicating at least one of a fluoroscopic image and a condition for collecting the fluoroscopic image,
evaluate image quality of the fluoroscopic image based on the fluoroscopy-related information, and
output identification information identifying whether to save the fluoroscopic image based on an evaluation result.

9. A method comprising:
acquiring fluoroscopy-related information indicating at least one of a fluoroscopic image and a condition for collecting the fluoroscopic image;
evaluating image quality of the fluoroscopic image based on the fluoroscopy-related information; and
outputting identification information identifying whether to save the fluoroscopic image based on an evaluation result.
